(19) United States
(12) Patent Application Publication
(10) Pub. No.: US 2020/0319208 A1
(43) Pub. Date: Oct. 8, 2020

(54) METHOD FOR QUANTIFYING PROTEIN AGGREGATES OF A PROTEIN MISFOLDING DISEASE IN A SAMPLE

(71) Applicant: Forschungszentrum Juelich GmbH, Juelich (DE)

(72) Inventors: Christian Zafiu, Vienna (AT); Dieter Willbold, Juelich (DE); Bettina Kass, Juelich (DE)

(21) Appl. No.: 16/758,048

(22) PCT Filed: Oct. 25, 2018
(86) PCT No.: PCT/DE2018/000309

§ 371 Date: Apr. 22, 2020

(30) Foreign Application Priority Data
Nov. 23, 2017 (DE) 10 2017 010 842.0

(51) Int. Cl. G01N 33/68; G01N 33/543

(52) U.S. Cl. CPC G01N 33/6896; G01N 33/543; G01N 2800/2835; G01N 2333/4709; G01N 2800/2828; G01N 2800/2821

(57) ABSTRACT

Provided herein is a method for quantifying protein aggregates of a protein misfolding disease in a sample, comprising: placing a capture molecule A on a substrate; selecting a complex sample comprising an aggregate of the protein misfolding disease; removing insoluble components from the sample; contacting the sample with capture molecule A on a part of the substrate; contacting a calibration standard with the capture molecule A on another part of the substrate, contacting at least one capture molecule B with both the aggregate of the sample and the calibration standard, wherein the capture molecule B can emit a detectable signal; comparing the signals of the at least one capture molecule B arranged on the sample assembly and on the calibration standard, wherein the steps do not have to be carried out successively. A related device, kit, and method for detecting protein aggregates are also provided.

### CROSS-REFERENCE TO PRIOR APPLICATIONS

This application is a U.S. National Phase application under 35 U.S.C. § 371 of International Application No. PCT/DE2018/000309, filed on Oct. 25, 2018, and claims benefit to German Patent Application No. 10 2017 010 842.0, filed on Nov. 23, 2017. The International Application was published in German on May 31, 2019 as WO 2019/101250 A1 under PCT Article 21(2).

### FIELD

The invention relates to a method for quantifying protein aggregates of a protein misfolding disease in a sample.

### BACKGROUND

Chromatographic separation methods such as size exclusion chromatography and separation methods based on ultracentrifugation methods are known from the prior art for separating proteins in a mixture from one another.

With the aid of different centrifugation techniques, for example, a sample can be fractionated with amyloid beta, so that different amyloid beta species are present in different fractions. These can then be analyzed by means of, for instance, ELISA, Western Blot, UV-VIS, mass spectroscopy or SDS-PAGE according to, for example, Funke et al (S. A. Funke, T. van Groen, I. Kadish, D. Bartnik, L. Nagel-Steger, O. Brener, T. Sehl, R. Batra-Safferling, C. Moriscot, G. Schoehn, A. H. C. Horn, A. Muller-Schiffmann, C. Korth, H. Sticht, D. Willbold. Oral Treatment with the D-Enantiomeric Peptide D3 Improves the Pathology and Behavior of Alzheimer's Disease Transgenic Mice. ACS Chem. Neurosci. (2010), 1, 639-648).

From Sehlin et al. (Dag Sehlin, Hillevi Englund, Barbro Simu, Mikael Karlsson, Martin Ingelsson, Fredrik Nikolajeff, Lars Lannfelt, Frida Ekholm Pettersson. 2012. Large Aggregates Are the Major Soluble Aβ Species in AD Brain Fractionated with Density Gradient Ultracentrifugation. Plos one, Vol. 7, e32014) it is known that large aggregates are the essential source of amyloid beta species by density gradient centrifugation fractionated Alzheimer's dementia brains. An ELISA method is recited for quantification.

According to Ward et al. (Robin V. Ward, Kevin H. Jennings, Robert Jepras, William Neville, Davina E. Owen, Julie Hawkins, Gary Christie, John B. Davis, Ashley George, Eric H. Karran and David R. Howlett. 2000. Fractionation and characterization of oligomeric, protofibrillar and fibrillar forms of β-amyloid peptide. Biochem. J. 348, 137-144), it is known that after density gradient centrifugation an immunoassay with the monoclonal antibody mAb158 was applied to quantify amyloid beta aggregates.

The disadvantage is that with the disclosed antibodies in the used Elisa method a highly sensitive quantification of amyloid beta is not possible from complex samples and mixtures. For this purpose, the cited publications only give an indication of amyloid beta up to the micromolar range. These methods are therefore not sensitive. Even the most sensitive Elisa methods only detect proteins up to a concentration of approximately 100 picomolar. From the present point of view, this seems not to be sufficient in order to also check the minor effects of a potential active substance for the therapy of Alzheimer's dementia for its effectiveness.

Certain embodiments of the invention provide a highly sensitive method and a device for quantifying individual protein aggregates in a complex sample or a mixture of samples and to specify further applications of the method.

### SUMMARY

Provided herein is a method for quantifying protein aggregates of a protein misfolding disease in a sample, comprising: a) placing, on a substrate, a capture molecule A for a monomer of the protein misfolding disease on a substrate; b) selecting a complex sample comprising an aggregate of the protein misfolding disease, wherein the aggregate has epitopes of the monomer at the surface of the aggregate; c) removing insoluble components from the sample; d) contacting the sample according to step c), with the capture molecule A on a part of the substrate, wherein the sample comprises the aggregate of the protein misfolding disease, and wherein the monomer contained therein is arranged on the capture molecule A; e) contacting and arranging thereon a calibration standard with the capture molecule A on another part of the substrate, wherein a defined number of monomers of the protein misfolding disease to be detected is arranged on the surface of the calibration standard; f) contacting at least one capture molecule B for the monomer of the protein misfolding disease with both the aggregate of the sample and the calibration standard and arranging the capture molecule B on the monomer of the protein misfolding disease, wherein the capture molecule B can emit a detectable signal; and g) comparing the signal of the capture molecule B arranged on the sample assembly with the signal of the capture molecules B arranged on the calibration standard to quantify the sample assembly, wherein steps a) to g) do not have to be carried out successively.

Also provided are a related device, kit, and method for detecting protein aggregates.

### BRIEF DESCRIPTION OF THE DRAWINGS

FIG. 1: Aggregate assay result of two mouse brain samples fractionated and homogenized by density gradient centrifugation (GGZ).

FIG. 2: Silver staining of the proteins in the individual fractions of the brain homogenate of an APP_{swe}/PS1ΔE9 transgenic mouse.

FIG. 3: Western blot of amyloid beta proteins in the individual fractions of the brain homogenate_{swe}/PS1ΔE9 transgenic mouse.

### DETAILED DESCRIPTION

The invention relates to a method for quantifying protein aggregates of a protein misfolding disease in a sample, characterized by the steps of:
a) A capture molecule A for the monomer of the protein misfolding disease is placed on the substrate;

In particular, protein misfolding diseases of humans and animals come into consideration as a protein misfolding disease.

TABLE 1
Protein and associated protein misfolding disease

| Protein monomer or epitope | Protein misfolding disease |
|---|---|
| Amyloid beta | Alzheimer's dementia |
| Prion protein | Prion diseases |
| Serum amyloid A protein | AA Amyloidosis |
| IgG light chain | AI Amyloidosis |
| AApoAI | AApoAI amyloidosis |
| AApoAII | AApoAII amyloidosis |
| ATTR | ATTR Amyloidosis |
| DISC1 | DISC1opathies |
| FUS | FUS proteinopathies |
| IAPP | Diabetes mellitus type 2 |
| SOD1 | Amyotrophic lateral sclerosis |
| α-Synuclein | Synucleinopathies |
| Tau | Tauopathies |
| TDP-43 | TDP-43 proteinopathies |
| Huntingtin | Huntington's disease |
| Lysozyme | Familial visceral amyloidosis |

In one embodiment, the material of the substrate is selected from the group comprising or consisting of plastic, silicon and silicon dioxide. In a preferred embodiment, glass is used as the substrate.

In one embodiment, a microtiter plate with its many reaction chambers is used as substrate. These can be advantageously read, e.g. microscopically. The capture molecule A is preferably arranged on the surface of the reaction chambers.

In an embodiment, a substrate having a hydrophilic surface is used for this purpose. In another embodiment, this is achieved by the application of a hydrophilic layer, prior to step a), to the substrate. Consequently, the molecules of capture molecule A bind, in particular, covalently to the substrate or to the hydrophilic layer with which the substrate is loaded.

The hydrophilic layer is a biomolecule-repellent layer, so that the nonspecific binding of biomolecules to the substrate is advantageously minimized. The molecules of the capture molecule A are preferably immobilized, preferably covalently, onto this layer. These are affine to a feature in the protein aggregate.

In one embodiment, the hydrophilic layer is selected from the group comprising or consisting of polyethylene glycol, poly lysine, preferably poly D lysine, and dextran or derivatives thereof, preferably carboxymethyl-dextran (CMD). Derivatives within the meaning of the invention are compounds which differ in some substituents from the parent compounds, the substituents being inert to the method according to the invention.

In one embodiment of the invention, the surface of the substrate is first hydroxylated and activated with amino groups prior to application of the hydrophilic layer. In one alternative, this activation with amino groups is carried out by bringing the substrate into contact with APTES (3-aminopropyl-trietoxy silane) or with ethanolamine.

In order to prepare the substrate for coating, one or more of the following steps can be carried out:
washing a substrate of glass or a glass carrier in an ultrasonic bath or plasma cleaner; alternatively, incubating in 5 M NaOH for at least 3 hours,
rinsing with water and subsequently drying under nitrogen,
dipping into a solution of concentrated sulfuric acid and hydrogen peroxide at a ratio of 3:1 for the activation of the hydroxyl groups,
rinsing with water to a neutral pH, subsequently washing with ethanol and drying under a nitrogen atmosphere,
dipping into a solution of 3-aminopropyltrietoxysilane (APTES) (1-7%), preferably in dry toluene or in a solution of ethanolamine,
rinsing with acetone or DMSO and water, and drying under a nitrogen atmosphere.

In an embodiment, the substrate is brought into contact with APTES in the gas phase; the pretreated substrate, if necessary, is therefore vaporized with APTES.

For coating with dextran, preferably carboxymethyl-dextran (CMD), the substrate can be incubated with an aqueous solution of CMD in a concentration of 10 mg/ml or 20 mg/ml and optionally N-ethyl-N-(3-dimethylaminopropyl)carbodiimide (EDC), (200 mM) and N-hydroxysuccinimide (NHS), (50 mM) and subsequently washed.

In one embodiment, the carboxymethyl-dextran is covalently bonded to the glass surface, which was first hydroxylated and, in particular, functionalized with amino groups.

Microtiter plates, preferably with a glass bottom, can be used as the substrate. Since the use of concentrated sulfuric acid is not possible when polystyrene frames are used, the glass surface is activated analogously in an embodiment of the invention.

In a further embodiment of the invention, the capture molecule A is covalently bound to the substrate.

The capture molecule A, which is affine to a feature of the protein aggregate to be detected, is immobilized, by way of example, on a hydrophilic layer, preferably covalently. This feature can be an epitope or a subsequence of a protein aggregate.

In an embodiment, only one type of capture molecule A is used. This advantageously has the effect that a type of aggregate of a single protein misfolding disease can be detected sensitively quantitatively.

In one embodiment of the present invention, the capture molecule A, preferably an antibody, is immobilized on the substrate, optionally after activation of the CMD-coated carrier by a mixture of EDC/NHS (200 or 50 mM).

Remaining carboxylate end groups to which no molecules of the capture molecule A were bound can be deactivated. Ethanolamine is used to deactivate these carboxylate end groups on the CMD spacer. Prior to the application of the samples, the substrates or carriers are optionally rinsed with buffer.

In an embodiment, a monoclonal antibody directed against the monomer of the protein misfolding disease can be used as capture molecule A. In the case of Alzheimer's dementia, the monoclonal antibody Nab228 may, by way of example, be used and placed on the substrate.

In contrast to the capture molecule B described below, in an embodiment, the capture molecule A has no detection molecule or molecular parts which are suitable for detection in the method according to the invention.

The term “arrangement” as used herein comprises, but is not limited to, covalent bonds. In the case of the antibodies, these are specific.

In an embodiment, the method comprises step b).
b) A complex sample comprising the aggregate of protein misfolding disease is provided, wherein the aggregate comprises epitopes of the monomer at the surface of the aggregate;

The provision of a complex sample means, in particular, the selection of an already prepared sample which originates from a diseased animal and/or a diseased human having the protein misfolding disease or in which it is to be checked whether or not the corresponding aggregates are present.

For example, a sample from an animal is selected, in particular a transgenic mouse suffering from Alzheimer's dementia. The sample can be obtained after the death of the animal by preparing at least one brain half. Preparation means in particular the homogenization of brain tissue.

In this way, a sample obtained from a diseased animal is selected and examined for the presence of a protein misfolding disease.

However, the sample can also be a cell culture, or an organ taken from an animal or a human or a sample from a biopsy. The sample contains or is examined for endogenously formed peptide or protein aggregates of the protein misfolding disease.

The method relates in particular and particularly advantageously and surprisingly to the highly selective detection of a specific aggregate of a protein misfolding disease from a mixture of proteins and/or protein fragments in the same sample. A specifically detectable aggregate of a protein misfolding disease can advantageously be detected in a highly sensitive manner in the selected sample from a mixture with more than one protein or protein fragment.

In particular, the sample may contain more than 2, 3, 4, 5, 6, 7, 8, 9, or even more than 10, 20, 30, 40, 50, 60, 70, 80, 90 or even more than 100, 200, 300, 400, 500, 600, 700, 800, 900 or even more than 1000 different proteins or protein fragments. The sample may contain thousands of proteins and/or protein fragments. In this sense, it is referred to as a complex sample.

The term “complex sample” comprises in particular the complete homogenate with the complete soluble protein content, in particular the brain of a deceased animal or human.

In an embodiment, in step b) in particular the brain homogenate of an animal, in particular a transgenic mouse suffering from Alzheimer's dementia, is provided as a complex sample. This also comprises thousands of different proteins and/or protein fragments.

In an embodiment, in step b) a complex sample containing the amyloid beta aggregate of Alzheimer's dementia can thus be selected.

Without being bound to a particular theory, since, for some years, particularly the small, soluble Aβ aggregates (Aβ oligomers) are made responsible for being the main cause of the development and progress of Alzheimer's dementia, detecting active substance candidates for efficiency in order to reduce toxic aggregates in a highly sensitive manner even down to the femtomolar range or even a complete elimination can be achieved. If an active substance is examined for its ability to eliminate the aggregates, it is possible to detect these aggregates down to the femtomolar range. If oligomeric aggregates are no longer detected, the healing of Alzheimer's dementia or at least improvement of the course of the disease is detected by the method.

The higher molecular weight structures, such as the fibrils occurring in Alzheimer's dementia, are also detectable as an aggregate.

The same applies to the case in which an aggregate of another protein misfolding disease is to be detected.

In the simplest case, the term “quantifiable” as used herein can also be qualitatively interpreted by detecting the aggregate of the protein misfolding disease in a yes/no response.
c) Step c) involves removing the insoluble components from the selected sample.

Step c) therefore requires, for example, filtration or ultracentrifugation to remove the soluble components from the sample. Other methods are conceivable, which the person skilled in the art can also apply according to his or her expert knowledge.

In particular, density gradient centrifugation is preferred. This has the advantage that the sample is fractionated after step c).

A density gradient centrifugation is advantageously carried out, by which the sample is fractionated into up to 3, 4, 5, 6, 7, 8, 9, better into up to 10 and especially advantageously into 11, 12, 13, 14 and especially advantageously into up to 15 fractions.

This is an advantageous way of providing and using a specific fraction that differs significantly in its s-value from other components of the other fractions. Thus, a selection of certain fractions such as protofibrils or other precursors, such as oligomers, can be selected and further investigated.

The particles formed from the amyloid and/or aggregating peptides and/or proteins are thus separated from one another.

In this way, it is advantageous to obtain a plurality of fractions from the sample. The fractions contain the particles of amyloid and/or aggregating peptides and/or proteins, each having a particular aggregate sequence and form. This separation of the particles can advantageously be carried out by means of density gradient centrifugation according to the s value.

The fractionation of the amyloid and/or aggregating peptides and/or proteins present in the sample solution is carried out in a particularly advantageous manner by means of a density gradient centrifugation using, for example, Optiprep, Percoll, sucrose or an analogous density gradient material. The aggregates are separated according to size and, if necessary, shape (sedimentation coefficient). This method is particularly advantageous for aggregating Aβ(1-42) aggregates and tau aggregates.

Alternatively, size exclusion chromatography, which separates according to the hydrodynamic radius, can be used. Alternatively, fractionation is by means of asymmetric flow field flux fractionation, or by means of capillary electrophoresis. These methods are also advantageously suitable for calibration.

Other physical parameters of the aggregates can also be used as a basis for carrying out fractionation, e.g. the hydrodynamic radius of the particles. The fractions are spatially separated from one another, for example by pipetting off.

One is therefore not limited to a density gradient centrifugation. However, density gradient centrifugation has the advantage of providing all aggregates of amyloid and/or aggregating peptides and/or proteins originally present in the sample for further quantitative analysis.

The density gradient centrifugation itself is calibrated so that the fractions are exactly determined in terms of their s value. The term “exactly determined” therefore comprises a calibration step, by fractionation of molecules of known type and behavior. After fractionation, each fraction contains only one specific (i.e. known) type of conformer, e.g. oligomers or fibrils and so on, that are present in the protein misfolding disease.

In some embodiments, in step c) the sample can also pass through different preparation steps known to the person skilled in the art.

In an embodiment of the present invention, the sample is pretreated according to one or more of the following method steps before the sample is placed on the capture molecule A:
diluting with water or buffer,
treatment with enzymes, by way of example, proteases, nuclease, lipases,
centrifuging,
precipitation,
competition with probes to displace any antibodies present.

In some embodiments, the method requires step d).
d) The sample after step c), comprising the aggregate of protein misfolding disease, is brought into contact at a part of the substrate with the capture molecule A after step a) and the monomer contained therein and/or also the aggregate of the protein misfolding disease is arranged on the capture molecule A;

In some embodiments, the arrangement is specifically carried out on capture molecule A.

The arrangement is preferably performed specifically on a monoclonal antibody as capture molecule A, as described. This has the advantage that a very specific binding is established between the aggregate of the sample and its monomeric epitopes on the surface of the aggregate and the capture molecule A. This has the advantage that, for example, other proteins, especially aggregates of other protein misfolding diseases with comparable s-values, are excluded. Thus, only one specific aggregate is detected by the capture molecule A.

In some embodiments, the sample to be measured is brought into contact with the substrate prepared in this way and optionally incubated. Endogenous fluids or tissue can be used as the sample to be examined. In one embodiment of the present invention, the sample is selected from brain homogenate, cerebrospinal fluid (CSF), blood, plasma and urine. However, it can also be random samples of an organ (biopsy) or the homogenate of the entire organ, e.g. the brain.

In one embodiment of the present invention, the sample or the aggregate is arranged directly on the capture molecule A.

Non-specifically bound substances can be removed by at least a washing step.

In some embodiments, the method is continued with step e).
e) A calibration standard is brought into contact with another part of the substrate on the capture molecule A after step a) and arranged, wherein a defined number of monomers of the protein misfolding disease to be detected is arranged on the surface of the calibration standard;

In some embodiments, the calibration standard used is particles that are advantageously approximately the size of the aggregate of the protein misfolding disease. The size can be deduced from the literature without effort by the person skilled in the art.

In some embodiments, in step e) the calibration standard used is a particle which has a defined number of monomers on the surface which corresponds to the number of monomer epitopes of the aggregate to be detected.

This has the advantage of

1. improving the simulation of the protein misfolding disease aggregate through the calibration standard on the substrate because of the identical size of the calibration standard to the aggregate, and
2. a calibration is possible with regard to a later exact quantification of the aggregate with regard to the monomer epitopes actually present in the aggregate at the top surface of the aggregate, since the calibration standard has approximately as many epitopes as the aggregate.

By way of example, for the detection of amyloid beta aggregate of Alzheimer's dementia, a particle with a diameter of approximately 20 nm should be arranged as the calibration standard, since an amyloid beta aggregate can assume this size. Approximately 20-30 amyloid beta monomers should then be arranged, e.g. covalently arranged, on the surface of the calibration standard.

In some embodiments, in step e), a silica nanoparticle of approximately 20 nm is preferably used as the calibration standard and approximately 30 amyloid beta monomers are used on the surface. This corresponds to the size of the amyloid beta aggregates and the number of accessible monomer epitopes in the oligomer or in the aggregate.

The calibration standard can be synthesized for the recited purposes as follows, noting that use of the standard is not limited to the following methods:

### Method 1:

A) Providing an inorganic nanoparticle with approximately the size of the aggregate of the protein misfolding disease,
B) formation of free amino groups on the surface of the nanoparticle for functionalizing the nanoparticle surface to form an amine-functionalized nanoparticle,
C1) formation of free carboxyl groups on the free amino groups from step B) to form free carboxyl groups on the surface of the nanoparticles,
D1) activation of the free carboxyl groups from step C1), e.g. by the formation of NHS esters on the carboxyl group, and
E1) bonding free amines of the monomers or subcomponents of the monomers to the NHS esters from step D1).

### Alternative Method 2:

A) Providing an inorganic nanoparticle with approximately the size of the aggregate of the protein misfolding disease,
B) formation of free amino groups on the surface of the nanoparticle for functionalizing the nanoparticle surface to form an amine-functionalized nanoparticle,
C2) binding of Maleinimido spacer carboxylic acid to the free amino groups in step B),
D2) binding of monomers of the protein aggregate to the Maleinimido spacer carboxylic acids from step C2) via a sulfhydryl group at the free end of the monomers.
   Other methods are conceivable.

In some embodiments, step f) of the method according to the invention is then carried out.
f) A capture molecule B against the monomer of the protein misfolding disease is brought into contact both with the aggregate of the sample on the substrate and with the calibration standard and is arranged on the monomer of the protein misfolding disease, wherein the capture molecule B can emit a detectable signal;

The capture molecule B thus represents a probe. The term “capture molecule B” and “probe” are used synonymously.

On the other hand, the aggregate of protein misfolding disease to be detected from the sample is already arranged on capture molecule A and is thus immobilized on the substrate.

In one embodiment of the invention, the capture molecule A and the capture molecules B may have identical affine molecules or molecular parts. In another embodiment, different affine molecules or parts of molecules may be combined with different detection molecules or parts, or alternatively, different affine molecules or parts may be combined with identical detection molecules or parts.

It is also possible to use mixtures of various capture molecules B.

The use of a plurality of different probes coupled to different detection molecules B or molecule parts increases on the one hand the specificity of the signal (correlation signal), and on the other hand, this allows the identification of protein aggregates which differ in one or more features. This enables selective quantification and characterization of the protein aggregates. The capture molecule A and the capture molecule(s) B bind to the same epitope or to the same overlapping portion of an epitope of the monomer.

In some embodiments, in a further step, the protein aggregates immobilized on capture molecule A are marked with one or more probes, with capture molecule B, useful for further detection. As described above, the individual steps can also be performed in a different order according to some embodiments of the invention.

It can be advantageous to select one or more capture molecules B that bind to monomers of the protein aggregates, wherein the capture molecules B are also capable of emitting a specific signal only after binding to the aggregate.

For the purposes of the present invention, “quantitative determination” first means the determination of the concentration of the protein aggregates and thus also the determination of their presence and/or absence.

By suitable washing steps, excess capture molecules B which are not bound to the protein aggregates are removed. This allows the sensitivity to be further increased by reducing the background signal.

In an embodiment, these excess capture molecules B are not removed. This eliminates one washing step and there is no equilibrium shift towards dissociation of the protein aggregate-probe complexes or compounds. Due to the spatially resolved detection, the excess probes are not recorded during the evaluation.

In one embodiment, the binding sites of the protein aggregates are epitopes and the capture molecules and probes are antibodies and/or antibody parts and/or fragments thereof.

In one embodiment of the present invention, the capture molecule A and the capture molecule or capture molecules B differ.

For example, different antibodies and/or antibody parts and/or fragments can be used as capture molecules B. In a further embodiment of the present invention, capture molecule A and one or more capture molecules B which are identical to one another with the exception of the possible (dye) marking are used.

In a further embodiment of the present invention, at least two capture molecules B are used which contain, for example, different antibodies and optionally also carry different dye markings.

In each of the cases described above, only one kind of capture molecule A is used.

For detection purposes, the capture molecules B are characterized in that they preferably emit an optically detectable signal selected from the group consisting of fluorescence, bioluminescence and chemiluminescence emission and absorption.

In an embodiment, the capture molecules B as probes are thus marked with fluorescent dyes. The dyes known to the person skilled in the art can be used as fluorescent dye. In other embodiments, GFP (Green Fluorescence Protein), conjugates and/or fusion proteins thereof, and quantum dots may be used.

The capture molecule A has no probe function like the capture molecule B. The capture molecule A with a fluorescent dye can only be used for quality control of the surface, for example to prove the uniformity of the coating with capture molecule A. For this purpose, a dye is preferably used which does not interfere with the detection of the fluorescent dye of the probe on the protein aggregate. This enables subsequent control of the substrate structure and standardization of the measurement results.

The capture molecules or molecules B can be selected and used in such a way that the presence of individual protein aggregates features does not influence the measurement result.

In particular, a fluorescent, monoclonal antibody as capture molecule B can be brought into contact with the bound aggregate and the calibration standard against the monomer of the protein misfolding disease and arranged thereon.

Monoclonal antibodies can thus be selected in particular as capture molecule A and as capture molecule(s) B. This has the advantage that a sufficient sensitivity and strength of the arrangement is given to the aggregate and/or calibration standard.

A mixture of monoclonal antibodies such as mAb IC16 labeled CF-633 and Nab228 labeled CF-488 can be used as capture molecule B.

Molecules that bind to the same target region of the protein misfolding disease monomer should be selected as capture molecule A after step a) and capture molecule(s) B after step f). This has the particularly advantageous effect that monomers in the sample can no longer be bound by capture molecule(s) B, since the target region is already occupied by capture molecule A.

By way of example, in an embodiment, in the case of amyloid beta, the capture molecule A after step a) and the capture molecule B/antibody after step f) can both bind to amino acids 3-8 (viewed from the N′ end). If, for example, a sample with amyloid beta 1-42 aggregate is present on the capture molecule A of the substrate, the capture molecule B can only be bound to amyloid beta after step f) if further epitopes are present, i.e. an aggregate with further surface epitopes is bound.

It should be understood that these embodiments are merely exemplary in nature and are not intended to be limiting.

The method advantageously discriminates monomers from themselves, which are no longer rejected.

In some embodiments, step g) of the method can be carried out as follows:
g) The signal of the capture molecules B on the sample aggregate is compared with the signal of the capture molecules B arranged on the calibration standard for quantification of the sample aggregate.

Step g) then requires the detection of the signal, in particular a fluorescence signal, which is emitted by the fluorescent monoclonal antibody, for example, as capture molecule B. For example, a TIRF (Total Internal Reflection Fluorescence) system can be used for this purpose.

In one embodiment, a spatially resolved determination of the probe signal, that is to say a spatially resolved detection of the signal emitted by the probe, takes place. Accordingly, in this embodiment of the invention, methods based on a non-spatially resolved signal, such as ELISA or sandwich ELISA, are excluded.

A high spatial resolution is very advantageous for detection. In one embodiment of the method according to the invention, so many data points are collected that they allow the detection of a protein aggregate in front of a background signal which is caused, for example, by device-specific noise, other unspecific signals or non-specifically bound probes. In this way, as many values (read-out values) are read out as there are spatially resolved events (pixels). The spatial resolution determines each event against the respective background and thus represents an advantage over ELISA methods without a spatially resolved signal.

In one embodiment, the spatially resolved determination of the probe signal is based on total internal reflection fluorescence microscopy (tirfm) and the examination of a small volume element compared to the volume of the sample, in the range from a few femtoliters to below a femtoliter, or a volume range above the contact surface of the capture molecules with a height of 500 nm, preferably 300 nm, particularly preferred 250 nm, in particular 200 nm.

The immobilized and marked protein aggregates are detected by means of imaging the surface, e.g. using laser scanning microscopy. The highest possible spatial resolution determines a high number of pixels, whereby the sensitivity as well as the selectivity of the method can be further increased, since structural features can also be mapped and analyzed. Thus, the specific signal in front of the background signal of e.g., non-specifically bound probes increases.

Detection preferably takes place, for example, with spatially resolving fluorescence microscopy by a TIRF microscope, as well as the corresponding superresolution variants thereof, such as STORM and/or dSTORM.

In one embodiment of the present invention, a laser focus, such as is used in laser scanning microscopy, or an FCS (fluorescence correlation spectroscopy system) is used for this purpose as well as the corresponding super-resolution variants, such as STED, PALM or SIM.

Again, in contrast to ELISA, these methods result in as many read out values as there are spatially resolved events (e.g. pixels). Depending on the number of different probes, this information is advantageously multiplied. This multiplication applies to each detection event and leads to an information gain since it discloses further properties, e.g. a second feature, via protein aggregates. As a result of such a structure, the specificity of the signal can be increased for each event.

For evaluation, the spatially resolved information, e.g. the fluorescence intensity, of all probes used and detected is used in order to determine, for example, the number of protein aggregates, their size and their features.

For example, algorithms for background minimization and/or intensity threshold values can also be used for further analysis as well as pattern recognition.

Further image analysis options include, for example, the search for local intensity maxima in order to obtain from the image information the number of protein aggregates detected and also to be able to determine the particle sizes.

In some embodiments, washing steps may be carried out according to steps a), d), e) and f).

It is understood that the step sequence a) to g) as described herein serves merely for clarification and does not depict a sequence of steps that follow one another in time. Step b) and c) can be carried out, for example, before step a).

By way of example, in another embodiment of the method, the protein aggregates are brought into contact with the capture molecules B and arranged before the aggregates are brought into contact with the capture molecules A, thus immobilizing protein aggregates marked with probes on the substrate.

In an embodiment, the method can then be carried out, for example, as follows:

Method for quantifying protein aggregates of a protein misfolding disease in a complex sample,
characterized by the steps:
a) A capture molecule A is placed on a substrate against the monomer of the protein misfolding disease;
b) a complex sample comprising the aggregate of the protein misfolding disease is selected, wherein the aggregate has epitopes of the monomer at the surface of the aggregate;
c) the insoluble components are removed from the sample;
d) a calibration standard is brought into contact with the capture molecule A after step a) on a part of the substrate and arranged, wherein a defined number of monomers or parts of the monomers which have the epitope of the aggregates of the protein misfolding disease to be detected are present on the surface of the calibration standard;
e) at least one capture molecule B against the monomer of the protein misfolding disease is brought into contact with the aggregate of the sample and placed on the monomer of the protein misfolding disease, wherein the capture molecule or molecules B can emit a detectable signal;
f) the capture molecule or molecules B on the aggregate of the protein folding disease are brought into contact with the capture molecule A on a part of the substrate and the calibration standard on another part of the substrate, and the aggregate is arranged on the capture molecule A and on the calibration standard.
g) The signal of the capture molecules B on the sample aggregate is compared with the signal of the capture molecules B arranged on the calibration standard for quantification of the sample aggregate.

Thus, the capture molecules or molecules B can be bound to the aggregate before it is brought into contact with capture molecule A and immobilized on the substrate.

Any sequence is also possible here, e.g. steps b), c) and e) can be carried out before all other steps.

In a further embodiment of the method, the sample is chemically fixed with the capture molecule B after contacting the protein aggregates, e.g. by formaldehyde.

Surprisingly, it was discovered in the course of the invention that the method showed a very high sensitivity in the detection of aggregates in complex samples such as mouse brain homogenate and is insensitive to endogenously present monomers.

It is also particularly surprising that despite the complexity of the sample, no interfering signals were detected for samples from wild type mice. This shows that there are no interfering background signals. In wild type animals there is no human Abeta 1-42, therefore the antibody does not react here.

The method comprises aggregates of the respective protein misfolding disease in the femtomolar range, in particular up to a range of 1000-500 fM, particularly advantageously 100-500 fM, in particular 50-100 fM and 5-10 fM. Since it is not an ELISA-bound method, the detection is improved by a factor of up to 10,000 compared to this method.

The comparison of the defined number of epitopes on the calibration standard advantageously allows an exact quantification of the epitopes in the aggregate of the protein misfolding disease.

In an embodiment, a monoclonal antibody as capture molecule A in step a) and as capture molecule(s) B in step f) can be used which comprise, for example, amyloid beta 3-8 as the identical target region of the monomer. The above-mentioned purpose of no longer binding monomers of the sample is thereby achieved.

The method is therefore particularly suitable for detecting aggregates of protein misfolding diseases, since no monomer of the protein misfolding disease can be detected.

Surprisingly, the invention also solves the problem even in a complex matrix, such as brain homogenate with thousands of different proteins and protein fragments. The method thereby detects even very small amounts of protein aggregates even in the presence of its monomer, since it cannot be bound by capture molecule(s) B.

A further object that is achieved is that heterogeneous protein aggregates consisting of more than one type of protein can also be uniquely identified as aggregates.

A device for quantifying protein aggregates of a protein misfolding disease in a complex sample is also provided. This comprises a substrate on which a capture molecule A is arranged for a monomer of a protein misfolding disease. A particle with a defined number of monomers of the protein misfolding disease which corresponds to the number of epitopes in the aggregate to be detected is arranged as a calibration standard on a part of the capture molecule A on the substrate. Another part of the capture molecule A on another part of the substrate provides the binding sites for monomers of the protein misfolding disease aggregate from the complex sample.

The device comprises the calibration standard with which it is advantageously possible to quantify the aggregates up to the femtomolar range.

In some embodiments, the device is preferably characterized in that a particle with the size of the aggregate to be detected is arranged as calibration standard.

In an embodiment, the device is particularly characterized by a silica nanoparticle as a calibration standard.

In an embodiment, the device is particularly advantageous as a microtiter plate, wherein the microtiter plate has at least one reaction chamber as part of the substrate, on the bottom of which a calibration standard arranged on capture molecule A is arranged and has at least one further reaction chamber as part of the substrate, on the bottom of which capture molecule A is arranged for the aggregate of protein misfolding disease to be detected.

Further provided is a kit for quantifying aggregate of a protein misfolding disease, comprising:
a substrate on which a capture molecule A is arranged for a monomer of a protein misfolding disease and a calibration standard with a defined number of monomers of the protein misfolding disease is arranged on a part of capture molecule A; and
Separate therefrom is a capture molecule B on the substrate for the monomer of the protein misfolding disease, wherein the capture molecule A and the capture molecule B bind to the same target region of the monomer of the protein misfolding disease.

In certain embodiments, mixing trays and buffers for the capture molecule B are optionally present.

Further provided is a kit containing one or more of the following components:
substrate, optionally with a hydrophilic surface,
at least one capture molecule A,
alternatively: Substrate with capture molecule A, and/or calibration standard
capture molecule(s) B,
solutions,
calibration standard,
buffer.

The compounds and/or components of the kit of certain embodiments of the present invention may be packaged in containers, optionally with/in buffers and/or solution.

In certain embodiments, some components may be packaged in the same container. Additionally or alternatively, one or more of the components could be absorbed on a substrate as a solid carrier, such as a glass plate, a chip or a nylon membrane, or on the well of a microtiter plate. The substrate then comprises such a microtiter plate.

Further, in some embodiments, the kit may include instructions for use of the kit for any of the embodiments.

In a further embodiment of the kit, the capture molecules described above are already immobilized on the substrate. In addition, the kit may contain solutions and/or buffers. To protect the coating and/or the capture molecules immobilized on it, they can be covered with a solution or a buffer.

Further provided is the use of the method according to the invention for detecting protein aggregates in any sample for quantification and thus titer determination of protein aggregates.

The use of a device or kit as well as the expansion of the method are provided for the quantitative detection of the concentration of aggregate of a protein misfolding disease.

The use of the device or kit thus makes it possible to carry out a method that is sufficiently sensitive to detect an aggregate of a protein misfolding disease in complex samples such as homogenised brain at the end of an (animal) experiment.

By way of example, density gradient centrifugation can be used to examine any desired fraction for changes in concentration with or without addition of an active substance. In an embodiment, the method provides a method step which allows more than one fraction from density gradient centrifugation to be examined for changes in concentration or also for changes in aggregate size or other parameters.

Density gradient centrifugation preferably yields more than 3, 4 or more than 5 fractions, which can be examined both quantitatively and qualitatively, and not only with regard to changes in concentration. The term “desired fraction” in the sense of the method comprises, but is not limited to, such fractions which, before separation, also contained aggregating or aggregated peptides and/or protein components, in particular toxic oligomers.

The method is not causally directed to this, although it is understood that an active substance may be added to the sample comprising the amyloid and/or aggregating peptides and/or proteins of different aggregate number and form. Or it is an organ (e.g.: the brain) taken at the end of an animal study that serves as a sample. In this case, the samples of an animal treated with placebo can be compared with those of animals treated with active substance.

An active substance or the treatment with the active substance changes the dispersed distribution and thus the concentration of certain aggregates in the organ and thus in the homogenized sample. This concentration change is then quantitatively determined. The change is a prerequisite for the reduction or even for the complete elimination of certain toxic species with detectable aggregate or particle size. This means that the method detects the increase or decrease in the concentration of certain amyloid and/or aggregating peptides and/or proteins by changing the aggregate size distribution in the sample.

The composition of amyloid and/or aggregating peptides and/or proteins with different aggregation size and form is thus altered during the treatment under the influence of the active substance. Other particle sizes increase or remain constant under the influence of the active substance.

This makes it advantageous to quantitatively detect the change in concentration of amyloid and/or aggregating peptides and/or proteins, each with a specific size, with or without the influence of an active substance. The influence of the active substance on the distribution of aggregates of amyloid and/or aggregating peptides and/or proteins in the respective fraction can be quantitatively determined by comparison with controls without the active substance. This provides a means, inter alia, of the effectiveness of the active substance in terms of its ability to eliminate certain species during the treatment phase, e.g. toxic oligomers.

It is also possible to examine only a single fraction in this way. An embodiment of the invention provides a means of the ability of the active substance to quantitatively change the specific conformers from the fraction, e.g. to eliminate toxic oligomers in the animal model.

In one embodiment of the invention, the change in the shape distribution of the peptides and/or proteins under the influence of an active substance is also examined, preferably by fluorescence microscopic methods.

This allows the optional detection of a molecular active substance activity in the animal model. Of great advantage is the fast and reliable examination at the end of an animal study, which quantitatively proves the reduction of aggregated Aβ (A-Beta) as a function of the treatment with an active substance down to the femtomolar range.

The particularly advantageous combination of fractionation based on density gradient centrifugation and concentration determination by fluorescence microscopy, in particular laser scanning microscopy and TIRF microscopy, has thus led to the development of a method which is particularly sensitive in quantifying the influence of potential active substances on the proportion of toxic oligomer species of a Aβ(1-42) peptide or other peptides and/or proteins, since it does not measure the monomer form.

Optionally, in an embodiment, an active substance is employed in an animal study and tested for its dose-dependent influence on the particle size distribution of the sample in vivo as a function of a control. The method is particularly advantageous for screening potential active substances against Alzheimer's dementia (AD) based on the modulation of the toxic amyloid-ß (Aß) oligomers under the influence of the active substance. It is particularly advantageous if these studies are accompanied by behavioral examinations of the animals.

In an embodiment, the method according to the invention also provides a comprehensive, quantitative result with regard to the changing particle or aggregate size distribution of amyloid and/or aggregating peptides and/or proteins under the influence of the active substance. Thus, promising active substances, e.g. for a therapy of Alzheimer's dementia, which is supposed to reduce the concentration of soluble toxic components such as the Aβ oligomers, are examined for their effect in the animal model.

The method is not limited thereto. Furthermore, without limiting the method, this also allows the determination of whether the active substance leads to an increase in other potentially toxic or desired species in the animal model. The method is preferably used to identify active substances which, according to current knowledge, do not lead to an increase in other toxic constituents. To this end, several of the fractions obtained are particularly advantageously examined for changes in the concentration of the building blocks in accordance with the invention.

A comparison of the control with the sample containing an active substance or a natural ligand allows a reproducible and rapid determination of the active substance effectiveness with regard to the elimination and reduction of certain species such as oligomers and thus an estimation of its effect in an animal model and later in the clinical test phases.

It is conceivable that a plurality of potential active substances can be quantified quickly and reproducibly with the method according to the invention with respect to their influence on the particle size distribution of amyloid and/or aggregating peptides and/or proteins in a sample. In some embodiments, the sample can be of synthetic nature. However, in other embodiments, natural active substance or samples taken can also be examined in this way.

The invention is not yet limited to the previous embodiments. Instead, the method can also be carried out in a simplified version as follows:

Method for detecting protein aggregates of a protein misfolding disease in a sample comprising the steps of:
a) a complex sample comprising the aggregate of protein misfolding disease is selected, wherein the aggregate has epitopes of the monomer or detectable parts thereof at the surface of the aggregate;
b) the sample of step a) comprising the aggregate of protein misfolding disease is contacted with the substrate and the monomer contained therein is placed on the substrate;
c) at least one capture molecule B is brought into contact with the aggregate of the sample as a probe for detection against the monomer of the protein misfolding disease and is placed on the monomer of the protein misfolding disease, wherein the capture molecule B can emit a detectable signal.

In one embodiment of this method, a capture molecule A is arranged on the substrate against the monomer of the protein misfolding disease before step a) and in step b) the monomer of the aggregate is arranged on capture molecule A.

In a further embodiment of the invention, a sample is chosen for this purpose from which the insoluble components have been removed in advance.

In a further advantageous embodiment of the invention, a calibration standard is arranged on another part of the substrate or on another part of the capture molecule A, wherein an exactly defined number of monomers of the protein misfolding disease to be detected is arranged on the surface of the calibration standard.

In a particularly advantageous embodiment of this invention, the signal of the capture molecules B arranged on the sample aggregate is compared with the signal of the capture molecules B arranged on the calibration standard for quantifying the sample aggregate.

It is understood that further advantageous embodiments or effects will arise if the above-mentioned features of the special method with steps a) to g) is applied to the simplified method according to steps a) to c).

Thus, in the simplest case, the invention is the detection of an aggregate of a protein misfolding disease on a substrate with a corresponding probe, the capture molecule B.

### Exemplary Embodiments

The invention is explained in more detail below with reference to three exemplary embodiments and the accompanying figures, without this being intended to restrict the invention.

The calibration standard is provided in detail as follows:

### Step a Preparation of the Inorganic Nanoparticle.

200 ml of ethanol, 3.8 ml of 30% ammonium hydroxide, 3.5 ml of deionized water and 4.4 ml of tetraethoxysilane are constantly stirred in a round bottom flask for 2 days. The reaction product is silica nanoparticles having a diameter of about 20 nm. The separation is determined by transmission electron microscopy and corresponds to the amyloid beta aggregate. The yield is determined by evaporation of the solvent and weighing.

Step B: Surface Modification with Primary Amines to Form Free Amino Groups.

45 ml of the reaction solution from step A are mixed with 10 μl of glacial acetic acid and 165 μl of 3-aminopropyl triethoxysilane and stirred for four hours. The particles are then purified by centrifugation and resuspension in ethanol several times. The yield is determined by evaporation of the solvent and weighing.

Step C1: Surface Modification with Carboxyl Functionalities.

50 ml of the purified particles from step B are centrifuged, absorbed into 50 ml dimethylformamide and transferred to a round-bottomed flask. After adding 5 mmol of succinic acid anhydride to the solution, the solution is stirred under an argon atmosphere for one hour at 90° C. and heated. After the time elapsed, the solution continues to be stirred for 24 hours. The carboxylated particles are cleaned by centrifugation and resuspension in deionized water and the yield is determined by evaporation of the solvent and weighing.

Step C2: Surface Modification with Maleinimido Functionalities.

200 pmol particles from step B are added to 1 ml 100 mM 2-(N-morpholino)ethanesulfonic acid buffer at pH 6 with 10 volume % dimethylformamide and 40 μmol 1-ethyl-3-(3-dimethylaminopropyl)carbodiimide, 10 μmol N-hydroxysuccinimide, 40 μM 6-Maleimide hexanoic acid and mixed for one hour. The particles are purified by repeated centrifugation and resuspension in the above-mentioned buffer.

### D1: Bioconjugation of Amyloid Beta (1-42) on Carboxy Silica Nanoparticles.

100 pmol of carboxylated particles are absorbed in 1 ml of deionised water and 20 μmol 1-ethyl-3-(3-dimethylaminopropyl)carbodiimide, 5 μmol N-hydroxysuccinimide are added and mixed for one hour. The activated particles are purified by centrifugation and resuspension in deionized water several times. Finally, the particle pellet is absorbed in phosphate buffer, added to 0.3 mg recombinant amyloid beta 1-42 peptide and shaken overnight. The bioconjugated particles are purified by centrifugation, resuspension in hexafluoropropanol and incubation for one hour. The solvent is then purified by repeated centrifugation and resuspension in deionised water. The amyloid beta (1-42) particles are purified by centrifugation and resuspension in deionized water and the yield is determined by evaporation of the solvent and weighing. The number of epitopes is determined by a commercially available bicinchoninic acid test and related to the particle concentration.

D2: Bioconjuction of Amyloid Beta (Here 1-15) Peptides with C-Terminal Sulfhydryl Modification.

1 ml of the particles from step C2 are centrifuged and placed in a ml of 100 mM 2-(N-Morpholino)ethanesulfonic acid buffer at pH 6 with 10 volume % dimethylformamide and 5 mM ethylenediaminetetraacetic acid with 15 nmol of the peptide (sequence: DAEFRHDSGYEVHHQC, amyloid beta 1-15 with an additional cysteine modification) and shaken for 10 minutes. After the time elapsed, 50 μmol tris(2-carboxyethyl)phosphine is added to the solution and the solution is shaken overnight. The next day, the particles are cleaned by centrifugation and resuspension in deionized water and the yield is determined by evaporating the solvent and weighing. The number of epitopes is determined by a commercially available bicinchoninic acid test and related to the particle concentration.

The figures show:

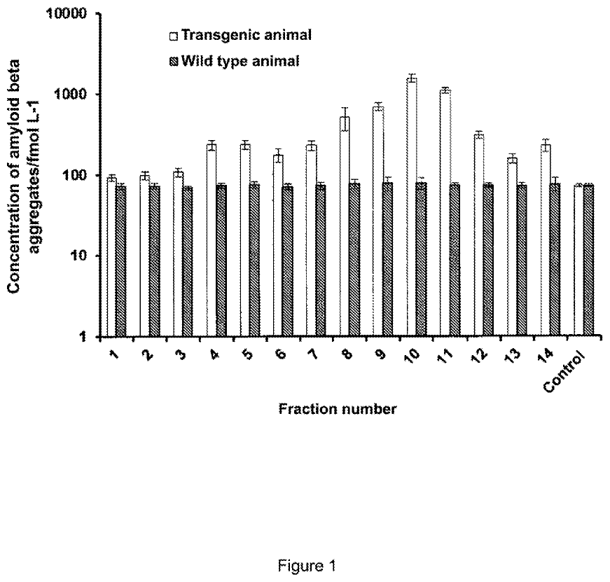

FIG. 1: Aggregate assay result of two mouse brain samples fractionated and homogenized by density gradient centrifugation (GGZ).

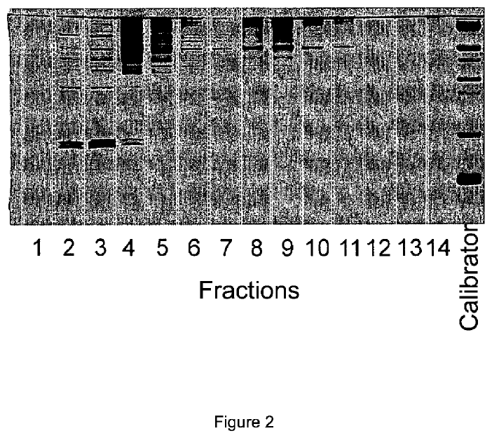

FIG. 2: Silver staining of the proteins in the individual fractions of the brain homogenate of an APP_{swe}/PS1ΔE9 transgenic mouse.

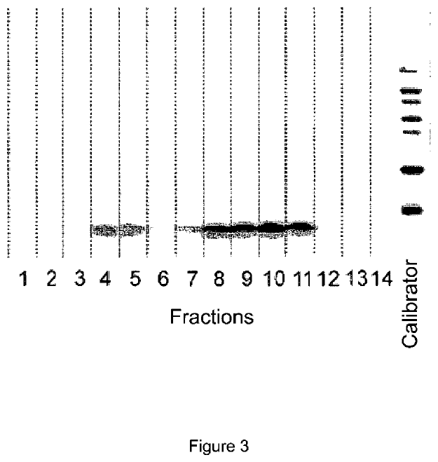

FIG. 3: Western blot of amyloid beta proteins in the individual fractions of the brain homogenate_{swe}/PS1ΔE9 transgenic mouse.

FIG. 1 shows the aggregate assay result of two mouse brain samples (whole hemisphere) fractionally homogenized by density gradient centrifugation (DGZ). Both mice were 24 months old at the time of organ withdrawal. The mouse designated as transgenic animal was an APP_{swe}/PS1ΔE9 double mutation that strongly expresses human amyloid beta. Wild type animals do not express human amyloid beta and are therefore not recognized in the assay by the use of antibodies that are directed against human amyloid.

The fractions are distinguishable and show the same distribution as the Western blot (FIG. 3). This means that the concentration shown in FIG. 1 corresponds approximately to the pattern in the Western blot (FIG. 3), but with the advantage of an exact quantitative statement by the method according to the invention.

The measurement was successful despite the strong background through the complex sample, as can easily be seen in FIG. 2 by myriad bands. In contrast to the Western blot, the method according to the invention allows the direct detection of specific amyloid beta aggregates without further separation (on the gel) and with the help of the calibration standard these are advantageously also absolutely quantifiable.

### Sample Preparation, Homogenization and Density Centrifugation

For homogenization, the right hemispheres were used and treated for 2×20 s at 6500 rpm (Precellys® 24, Bertin Instruments, Montigny-le-Bretonneux, France) with Tris buffer (pH 8.3, 20 mM Tris, 250 mM NaCl, and protease and phosphatase inhibitors (both Roche, Basel, Switzerland).

150 μl of the homogenate were centrifuged again at 1200 g for 10 min and 100 μl of the supernatant were applied to a density gradient. The density gradient consisted of 5 to 50% (w/v) Iodixanol (OptiPrep, Axis-Shield, Norway). After centrifugation (3 h, 259,000×g, at 4° C.) (Optima TL-100, Beckman Coulter, USA), 14 fractions (140 μl each) were removed from top to bottom, frozen in liquid nitrogen and stored at −80° C. until analysis.

### Plate Preparation and Measurement

For the assay, 384 well microtiter plates with 170 μm thick glass bottom (Sensoplate Plus, Greiner Bio-One GmbH, Frickenhausen, Germany) were initially prepared.

The glass bottom of the plate was silanized with APTES (99%; (3-aminopropyl) triethoxysilane; Sigma-Aldrich, Germany) by vapor deposition. Therefore the plate was stored in a desiccator over 5 μl of a solution consisting of 5% APTES in toluene (99% Sigma-Aldrich, Germany) in an argon atmosphere for 1 hour. The APTES solution was then removed, and the plate was dried under vacuum for 2 hours. A 2 mM succinimidyl carbonate-poly-(ethylene glycol)-carboxymethyl (MW 3400, Laysan Bio, Arab, USA) in dd H₂O was filled into the reaction chambers (RC) of the plate and incubated for 4 h. After the time elapsed, the reaction chamber was washed 3 times with water. The coating was then activated with 200 mM N-(3-dimethylaminopropyl)-N′-ethylcarbodiimides hydrochloride (98%; Sigma-Aldrich, Germany) and 50 mM N-hydroxysuccinimide (98%; Sigma-Aldrich, Germany) and incubated for 30 minutes. After washing three times with dd H₂O, 10 μg/ml capture antibody (capture molecule A) directed against the N-terminus of amyloid beta (Nab228 monoclonal antibody, Sigma-Aldrich, St. Louis, USA) were added to the reaction chamber in PBS and incubated for 1 hour. After washing three times with TBS+0.2% Tween (TBST) and TBS, the RC were blocked overnight with Smartblock solution (Candor Bioscience, Germany). The next day the plate was washed three times with TBS and samples and standards were applied to the plate as triplicate and incubated for 1 hour.

Brain homogenate samples were diluted ten times in TBS before being applied to the plate. The calibration standard for amyloid beta oligomers was Aβ1-42-SiNaPs (silica nanoparticles) with a diameter of 20 nm and about 30 epitopes (Aβ1-42), which were synthesized as described.

After washing three times with TBS, two different probe antibodies were used as capture molecules B (each 1.25 μg/ml) mAb IC16 marked with CF-633, (Sigma-Aldrich, Germany) and Nab228 (epitope Aβ1-10) marked with CF-488 (both Sigma-Aldrich, Germany). The probes were mixed and ultracentrifuged (100,000 g, 1 h, 4° C.) prior to addition to the plate and incubated for 1 hour.

After incubation, the reaction chamber was washed three times with TBS and the plate was sealed. Measurement was carried out in a Leica multi-line TIRF (total internal reflection fluorescence) system (AM TIRF Mc, Leica Microsystems, Wetzlar, Germany). The TIRF system was provided with an automated xyz stage and a 100×oil immersion objective (1.47 oil CORR TIRF Leica). The images were recorded consecutively at Ex/Em=633/705 nm and 488/525 nm with an exposure time of 500 ms and a gain of 800 for both channels. The TIRF penetration depth was set at 200 nm. The microscope took 5×5 images per RC in each channel. Each image consists of 1000×1000 pixels with a lateral resolution of 116 nm and a 14 bits intensity resolution.

Intensity limits were evaluated on the basis of the negative control in each channel. This limit value was then applied to all images and only those pixels which were at the same position in both channels (colocalized) above the intensity limits were counted. By evaluating the Aβ1-42-SiNaP standard, the number of colocalized pixels above the threshold values can be converted into oligomer concentrations.

FIG. 2 shows the silver staining of all proteins in the individual fractions of the brain homogenate of the APP_{swe}/PS11ΔE9 transgenic mouse.

For silver staining, 12 μl of each fraction (1 to 14) of DGZ was applied to SDS PAGE (sodium dodecyl sulfate polyacrylamide gel electrophoresis) in a 16.5% Tris Tricine gel at a constant current of 45 mA per gel for 110 min in a Mini PROTEAN Tetra Cell (Bio Rad, California, USA). After fixing the polyacrylamide gel overnight in 50% ethanol/10% acetic acid, the gel was incubated in 10% ethanol/5% acetic acid twice for 5 min. The gel was then incubated in a 4.7 mM Na₂CO₃, 4.6 mM K₃Fe(CN)₆, and 19 mM sodium thiosulfate Na₂S₂O₃ for 60 s and washed three times for 20 s with dd H₂O. The gel was then treated for 20 min in 12 mM AgNO₃ and washed again three times for 20 sec with dd H20. The coloration was developed in 280 mM Na₂CO₃ with 0.05% formalin (37% formaldehyde solution) until the desired intensity was achieved. Further development of the gel was stopped by treatment with 1% acetic acid for 5 min. The image was taken using a Chemiagen MP System (Bio Rad, California, USA).

FIG. 3 shows the Western blot of amyloid beta proteins in the individual fractions of the brain homogenate of the APP_{swe}/PS1ΔE9 transgenic mouse.

For silver staining, 12 μl of each fraction (1 to 14) of density gradient centrifugation was separated onto a SDS PAGE (sodium dodecyl sulfate polyacrylamide gel electrophoresis) in a 16.5% Tris Tricine gel at a constant current of 45 mA per gel for 110 min in a Mini PROTEAN Tetra Cell (Bio Rad, California, USA).

The proteins were transferred to a PVDF membrane with a pore size of 0.2 μm (Roti PVDF 0.2, Carl Roth, Germany) at 25 V and 1 A for 30 min. A Trans Blot Turbo Transfer System (Bio Rad, California, USA) was used for this purpose. The membranes were boiled in PBS for 5 min after the transfer. After cooling, the membranes were incubated in PBS and in TBS+Tween20 (TBST) for 5 min. Membranes were blocked with 10% skimmed milk powder/TBST (1 h, room temperature) and incubated with anti-Aβ antibody mAb IC16 (1:1000 in TBST overnight, 4° C.). The membranes were then washed 3 times for 10 min with TBST and incubated with an HRP-conjugated goat anti-mouse IgG (Thermo Fisher Scientific, Massachusetts, USA) (1:10000 in TBST). After washing three times for 10 min each with TBST, the protein bands were visualized in a ChemiDoc MP system (Bio-Rad, California, USA) using ECL Prime substrate (Amersham, Little Chalfont, United Kingdom).

It is understood that a simplified method results from the embodiment of the invention for detecting protein misfolding disease by omitting individual steps as recited herein in an abstract manner.

Thus, in the simplest case, the invention is the detection of an aggregate of a protein misfolding disease on a substrate with a corresponding probe, the capture molecule B, without the corresponding quantification step.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for quantifying protein aggregates of a protein misfolding disease in a sample,
comprising:
a) placing, on a substrate, a capture molecule A for a monomer of the protein misfolding disease;
b) selecting a complex sample comprising an aggregate of the protein misfolding disease, wherein the aggregate has epitopes of the monomer at the surface of the aggregate;
c) removing insoluble components from the sample;
d) contacting the sample according to step c), with the capture molecule A on a part of the substrate, wherein the sample comprises the aggregate of the protein misfolding disease, and arranging the monomer contained therein on the capture molecule A;
e) contacting and arranging thereon a calibration standard with the capture molecule A on another part of the substrate, wherein a defined number of monomers of the protein misfolding disease to be detected is arranged on the surface of the calibration standard;
f) contacting at least one capture molecule B for the monomer of the protein misfolding disease with both the aggregate of the sample and the calibration standard and arranging the capture molecule B on the monomer of the protein misfolding disease, wherein the capture molecule B can emit a detectable signal; and
g) comparing the signal of the capture molecule B arranged on the sample assembly with the signal of the capture molecules B arranged on the calibration standard to quantify the sample assembly, wherein steps a) to g) do not have to be carried out successively.

2. The method according to claim 1,
wherein
molecules which bind to the same target region of the monomer of the protein misfolding disease are selected as the capture molecule A according to step a) and as the capture molecule B according to step f).

3. The method according to claim 1,
wherein
monoclonal antibodies are selected as capture molecule A and/or as capture molecule B.

4. The method according to claim 1,
wherein
the capture molecule B comprises at least two monoclonal antibodies for the monomer of the protein misfolding disease.

5. A method according to claim 1,
wherein
the sample used in step b) comprises amyloid beta monomer of Alzheimer's dementia.

6. The method according to claim 5,
wherein
the capture molecule A in step a) is a monoclonal antibody and capture molecule B in step f) is a monoclonal antibody, and wherein each monoclonal antibody has amyloid beta 3-8 as an identical target region of the monomer.

7. The method according to claim 6,
wherein
the calibration standard in step e) is a particle containing the aggregate of the protein misfolding disease.

8. The method according to claim 1,
wherein
the calibration standard in step e) is a particle which has a defined number of monomers of the aggregate to be detected on the surface of the particle.

9. The method according to claim 1,
wherein
the calibration standard in step e) is a silica nanoparticle of approximately 20 nm in size with about 30 amyloid beta monomers on its surface.

10. The method according to claim 1,
wherein
the sample in step b) is a brain homogenate of a transgenic mouse with Alzheimer's dementia.

11. A device for quantifying protein aggregates of a protein misfolding disease in a complex sample, comprising a substrate on which a capture molecule A for a monomer of a protein misfolding disease is arranged and a particle is arranged on a part of the substrate on the capture molecule A as a calibration standard, wherein the particle comprises a defined number of monomers of the protein misfolding disease which corresponds to the number of monomer epitopes in the aggregate to be detected, and the capture molecule A provides binding sites for monomers of the protein misfolding disease from the complex sample on another part of the substrate.

12. The device according to claim 11,
wherein
the calibration standard is a particle with the size of the aggregate to be detected.

13. The device according to claim 11,
wherein
the calibration standard is a silica nanoparticle as a calibration standard.

14. The device according to claim 11,
wherein
the substrate is a microtiter plate, wherein the microtiter plate has at least one reaction chamber, on the bottom of which a calibration standard arranged on a capture molecule A is arranged, and the device has at least one further reaction chamber, on the bottom of which capture molecule A is arranged for the aggregate of the sample to be detected.

15. A kit for quantifying aggregate of a protein misfolding disease comprising:
a substrate on which a capture molecule A for a monomer of a protein misfolding disease is arranged, wherein a calibration standard is arranged on a part of the immobilized capture molecules A, and wherein the calibration standard comprises a defined number of monomers of the protein misfolding disease; and
at least one capture molecule B for the monomer of the protein misfolding disease, wherein the capture molecule A and the at least one capture molecule B bind to the same target region of the monomer of the protein misfolding disease.

16. A method for detecting the influence of an active substance on the concentration of an aggregate of a protein misfolding disease, comprising
contacting a sample with the substrate of the device of claim 11.

17. A method for detecting protein aggregates in a sample,
comprising:
a) selecting a complex sample comprising an aggregate of a protein misfolding disease, wherein the aggregate has epitopes of a monomer at the surface of the aggregate;
b) contacting the sample of step a) with a substrate, wherein the sample comprises the aggregate of the protein misfolding disease and wherein the monomer contained therein is placed on the substrate;
c) contacting a capture molecule B for the monomer of the protein misfolding disease with the aggregate of the sample on the substrate and placing the capture molecule B on the monomer of the protein misfolding disease, wherein the capture molecule B can emit a detectable signal.

18. The method according to claim 17,
wherein
a capture molecule A for the monomer of the protein misfolding disease is arranged on the substrate before step a) and in step b) the monomer is arranged on capture molecule A.

19. The method according to claim 17,
wherein
insoluble components in the sample were removed prior to the selecting step.

20. The method according to claim 18,
further comprising
contacting a calibration standard with the capture molecule A on a part of the substrate, wherein a defined number of monomers of the protein misfolding disease to be detected are arranged on the surface of the calibration standard.

21. The method according to claim 20,
further comprising
comparing a signal of capture molecules B arranged on the sample assembly with a signal of the capture molecules B arranged on the calibration standard to quantify the sample assembly.
